(19) United States
(12) Patent Application Publication
(10) Pub. No.: US 2025/0345077 A1
(43) Pub. Date: Nov. 13, 2025

(54) VENOUS THROMBUS TREATMENT CATHETER

(71) Applicant: TERUMO KABUSHIKI KAISHA, Tokyo (JP)

(72) Inventors: Takashi ITO, Shizuoka-shi (JP); Tetsuya FUKUOKA, Hadano-shi (JP)

(73) Assignee: TERUMO KABUSHIKI KAISHA, Tokyo (JP)

(21) Appl. No.: 19/277,088

(22) Filed: Jul. 22, 2025

Related U.S. Application Data
(63) Continuation of application No. PCT/JP2023/044013, filed on Dec. 8, 2023.

(30) Foreign Application Priority Data
Jan. 24, 2023 (JP) 2023-008650

(51) Int. Cl. A61B 17/22; A61B 17/00

(52) U.S. Cl. CPC A61B 17/22; A61B 17/00234; A61B 2017/00238; A61B 2017/00305; A61B 2017/22038; A61B 2017/22094

(57) ABSTRACT

A venous thrombus treatment catheter treats a lesion having an organized thrombus in a vein in a living body. The venous thrombus treatment catheter includes a first catheter and a second catheter that is movably inserted into a first lumen of the first catheter and has an outer diameter smaller than an outer diameter of an organized thrombus. The second catheter can protrude in the distal direction from a distal end part of the first catheter.

### CROSS-REFERENCES TO RELATED APPLICATIONS

This application is a continuation of International Application No. PCT/JP2023/044013 filed on Dec. 8, 2023, which claims priority to Japanese Patent Application No. 2023-008650 filed on Jan. 24, 2023, the entire content of both of which is incorporated herein by reference.

### TECHNOLOGICAL FIELD

The present invention generally relates to a venous thrombus treatment catheter.

### BACKGROUND DISCUSSION

Japanese Patent No. 6735320 discloses a treatment method for treating deep venous thrombosis (DVT). In this treatment method, an occlusion site (thrombus site) in a vein of a living body is identified on the basis of an intravascular image, a venous thrombus treatment catheter is penetrated into the occlusion site, and then a therapeutic balloon is delivered to the penetrated site and is subsequently expanded, thereby treating the occlusion site.

### SUMMARY

In general, in deep venous thrombosis, the diameter of the vein is larger than that of the normal blood vessel and is thinner than that of the normal blood vessel. However, an occlusion site (organized thrombus) may be formed between the enlarged vein portions, and the outer diameter of the occlusion site may be smaller than the outer diameter of the enlarged vein portion. When treating an occlusion site with a venous thrombus treatment catheter, it is required not to cause blood vessel perforation at a vein portion that is larger in diameter than a normal blood vessel and thinner than the normal blood vessel, and to form a penetrating portion penetrating the occlusion site after the catheter is caused to reach the occlusion site (organized thrombus) that is difficult to penetrate over a sufficient time. Therefore, the treatment time of the occlusion site by the venous thrombus treatment catheter becomes long.

(1) According to a first aspect, there is provided a venous thrombus treatment catheter for treating a lesion having an organized thrombus sandwiched in vein portions having diameters larger than a diameter of a normal blood vessel in an extending direction of a blood vessel of a vein in a living body, the venous thrombus treatment catheter including: a first catheter having a first lumen and puncturing a skin of the living body; and a second catheter that is movably insertable into the first lumen of the first catheter, has an outer diameter smaller than an outer diameter of the organized thrombus, and is capable of protruding in a distal direction from a distal end part of the first catheter.

According to the venous thrombus treatment catheter, when the venous thrombus treatment catheter is advanced into the vein and the lesion is punctured with the second catheter, the second catheter is suitably backed up along the advancing direction by the first catheter, so that the second catheter can easily penetrate the lesion (organized thrombus). As a result, the treatment time of the lesion by the venous thrombus treatment catheter can be shortened. As compared with the case of a single catheter, the outer diameter of the first catheter is increased to increase the rigidity, and the second catheter is guided by the first catheter to increase the support force, so that the second catheter can be stably moved straight along the traveling direction.

(2) In the venous thrombus treatment catheter according to (1), an outer diameter of the first catheter may be smaller than an outer diameter of the organized thrombus.

With this configuration, in a case where the venous thrombus treatment catheter is inserted into a vein from the peripheral side with respect to the lesion of the living body, the first catheter having a diameter smaller than that of the organized thrombus can penetrate the lesion (organized thrombus) together with the second catheter.

(3) In the venous thrombus treatment catheter according to (1), an outer diameter of the first catheter may be larger than an outer diameter of the organized thrombus.

With this configuration, the rigidity of the first catheter is enhanced, and the venous thrombus treatment catheter can be inserted into the vein from the central side where the diameter of the blood vessel is larger than that of the terminal side, so that the support force of the second catheter by the first catheter can be further enhanced.

(4) The venous thrombus treatment catheter according to any one of (1) to (3) may include a third catheter that is movably inserted into the first lumen of the first catheter, has a third lumen into which the second catheter is inserted, and is capable of protruding in a distal direction from a distal end part of the first catheter.

With this configuration, by guiding the outside of the second catheter by the third catheter, the deflection of the second catheter is suitably suppressed, and the support force of the second catheter is improved, so that the penetration of the second catheter with respect to the lesion can be improved.

(5) In the venous thrombus treatment catheter according to any one of (1) to (4), the second catheter may have a second lumen, and a guide wire may be inserted through the second lumen.

(6) A second aspect is a treatment method for treating, by a venous thrombus treatment catheter, a lesion having an organized thrombus sandwiched between vein portions having diameters larger than a diameter of a normal blood vessel in an extending direction of a blood vessel of a vein in a living body, in which the venous thrombus treatment catheter includes: a first catheter having a first lumen and puncturing a skin of the living body; and a second catheter that is movably inserted into the first lumen of the first catheter, has an outer diameter smaller than an outer diameter of the organized thrombus, and is capable of protruding in a distal direction from a distal end part of the first catheter, and the treatment method comprises: identifying the lesion with the organized thrombus; and causing the venous thrombus treatment catheter to advance along the vein, and causing at least the second catheter of the first and second catheters to penetrate the lesion after a distal end of the venous thrombus treatment catheter reaches the lesion.

According to this treatment method, when the venous thrombus treatment catheter is advanced into the vein and the lesion is punctured with the second catheter, the second catheter is suitably backed up by the first catheter, so that the second catheter can easily penetrate the lesion (organized thrombus). As a result, the treatment time of the lesion by the venous thrombus treatment catheter can be shortened. As compared with the case of a single catheter, by increasing the outer diameter of the first catheter to increase the rigidity and guiding the second catheter by the first catheter, the second catheter can be stably moved straight along the traveling direction.

(7) The treatment method according to (6) may perform, after forming a penetrating portion in the lesion by penetrating at least the second catheter into the lesion, inserting a treatment catheter into the penetrating portion of the lesion to treat the lesion.

According to this treatment method, by forming the penetrating portion with respect to the lesion by the second catheter of the venous thrombus treatment catheter, the treatment catheter can be inserted into the penetrating portion to reliably treat the lesion.

The venous thrombus treatment catheter includes the second catheter that is movably inserted into the first lumen of the first catheter, has an outer diameter smaller than the outer diameter of the organized thrombus, and is capable of protruding in the distal direction from the distal end part of the first catheter, whereby the second catheter is suitably backed up by the first catheter when the venous thrombus treatment catheter is advanced into the vein and the lesion is punctured with the second catheter. Therefore, the second catheter can easily penetrate the lesion (organized thrombus). As a result, the treatment time of the lesion by the venous thrombus treatment catheter can be shortened. As compared with the case of a single catheter, the outer diameter of the first catheter is increased to increase the rigidity, and the second catheter is guided by the first catheter to increase the support force, so that the second catheter can be stably moved straight along the traveling direction.

Another aspect involves a venous thrombus treatment catheter for treating, in a blood vessel of a vein in a living body, a lesion having an organized thrombus sandwiched between vein portions in an extending direction of the blood vessel, with the vein portions having diameters larger than a diameter of the blood vessel devoid of the thrombus. The venous thrombus treatment catheter comprises a combination of a first catheter having a first lumen extending throughout the first catheter and a second catheter having a second lumen extending throughout the second catheter, with the second catheter being positioned in the first lumen of the first catheter and being movable relative to the first catheter from a position in which a most distal end of the first catheter is on a distal side of a most distal end of the second catheter to a position in which the most distal end of the second catheter is on the distal side of the most distal end of the first catheter so that the second catheter is able to penetrate into the lesion. The first catheter has a distal end part that is tapered in a narrowing manner toward the most distal end of the first catheter so that the distal end part is configured to puncture skin of the living body, and the second catheter has a distal end part that is tapered in a narrowing manner toward the most distal end of the second catheter.

### BRIEF DESCRIPTION OF THE DRAWINGS

FIG. 1 is an explanatory view illustrating an initial state when treatment is performed using a venous thrombus treatment catheter according to an embodiment.

FIG. 2 is an explanatory view illustrating a state in which a second catheter of the venous thrombus treatment catheter penetrates a lesion.

FIG. 3 is an explanatory view illustrating a state in which a treatment unit of a treatment catheter is inserted into a penetrating portion of a lesion.

FIG. 4 is an explanatory view illustrating a case where a treatment is performed by inserting the venous thrombus treatment catheter into a central blood vessel portion.

### DETAILED DESCRIPTION

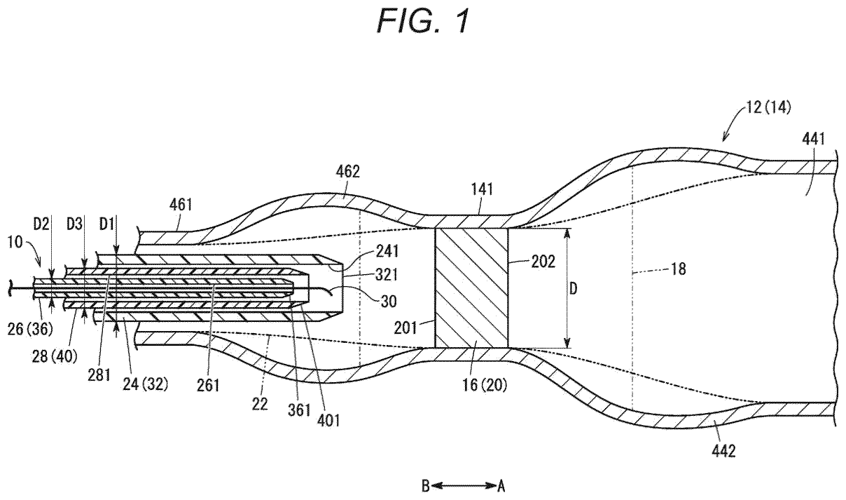

As illustrated in FIG. 1, a venous thrombus treatment catheter 10 according to the present embodiment, representing one example of the new venous thrombus treatment catheter disclosed here, is used, for example, for treatment of a lesion 16 (such as a stenosis or an occlusion portion) generated in a blood vessel 141 of a vein 14 in a living body 12. Specifically, the venous thrombus treatment catheter 10 is used to treat deep venous thrombosis (DVT, the lesion 16) in which an organized thrombus 20 in which a thrombus 18 is organized occurs in the blood vessel 141 of the vein 14.

The venous thrombus treatment catheter 10 is inserted into the blood vessel 141 of the vein 14 in the living body 12 and can advance along the blood vessel 141. The venous thrombus treatment catheter 10 is a catheter that advances toward the peripheral side (ankle side) or the central side (heart side) of the living body 12 along the blood vessel 141 with respect to the lesion 16 in the vein 14.

The venous thrombus treatment catheter 10 includes a first catheter 24 having a first lumen 241, a second catheter 26 movably inserted into the first lumen 241, a third catheter 28 disposed between the first catheter 24 and the second catheter 26, and a guide wire 30 inserted into a second lumen 261 of the second catheter 26.

The first catheter 24 has a tubular first catheter body 32 having the first lumen 241. The first catheter 24 is, for example, a sheath introducer. The first catheter body 32 is formed of a flexible resin material. The first catheter body 32 is percutaneously inserted into the blood vessel 141 of the vein 14 of the living body 12. A distal end part 321 of the first catheter body 32 has a tapered outer peripheral surface whose diameter (outer diameter) decreases in the distal direction. The first lumen 241 is disposed inside the first catheter body 32. The first lumen 241 extends along the first catheter body 32. The proximal end of the first catheter body 32 includes a hub (not illustrated), and the proximal end of the hub is opened. In a case where the venous thrombus treatment catheter 10 is inserted into the blood vessel 141, a dilator (not illustrated) may be inserted into the first lumen 241 of the first catheter 24, and a hub (not illustrated) may be a hemostasis valve.

An outer diameter D1 (outer peripheral diameter) of the first catheter body 32 is smaller than an outer diameter D of the organized thrombus 20 (lesion 16) in a cross section orthogonal to the extending direction of the blood vessel 141 (D1<D).

The second catheter 26 has a tubular second catheter body 36 having the second lumen 261. The second catheter 26 is, for example, a microcatheter or a support catheter. The second catheter body 36 is formed of a flexible resin material. An outer diameter D2 of the second catheter body 36 is smaller than the outer diameter D1 of the first catheter 24 (D2<D1). That is, the outer diameter D2 of the second catheter body 36 is smaller than the outer diameter D of the organized thrombus 20 in the cross section orthogonal to the extending direction of the blood vessel 141 (D2<D).

The second catheter body 36 may be inserted into the first lumen 241 of the first catheter 24 via the third catheter 28. The second catheter body 36 is provided movably along the first lumen 241. A distal end part 361 of the second catheter body 36 has a tapered outer peripheral surface whose diameter (outer diameter) decreases in the distal direction.

When the second catheter 26 moves along the first lumen 241 of the first catheter 24, the second catheter 26 is guided (supported) in the moving direction by the first catheter 24 via the third catheter 28. The distal end part 361 of the second catheter 26 can protrude in the distal direction from the distal end part 321 of the first catheter 24. The distal end part 361 of the second catheter body 36 is not made of a soft material such as a rubber material (elastomer material), but has hardness suitable for treatment of the lesion 16 (organized thrombus 20). The second lumen 261 is disposed inside the second catheter body 36. The second lumen 261 extends along the second catheter body 36. The second lumen 261 is a passage through which the guide wire 30 can be inserted and can pass.

The proximal end of the second catheter body 36 includes a hub (not illustrated), and the proximal end of the hub is opened. The guide wire 30 can be inserted into the second lumen 261 through the hub.

The third catheter 28 has a tubular third catheter body 40 having a third lumen 281. The third catheter 28 is, for example, an imaging catheter or a guiding catheter. The third catheter body 40 is formed of a flexible resin material. An outer diameter D3 of the third catheter body 40 is smaller than the outer diameter D1 of the first catheter 24 and larger than the outer diameter D2 of the second catheter 26 (D2<D3<D1). A distal end part 401 of the third catheter body 40 has a tapered outer peripheral surface whose diameter (outer diameter) decreases in the distal direction. The distal end part 401 of the third catheter body 40 is not made of a soft material such as a rubber material (elastomer material), and has hardness suitable for treatment of the lesion 16. In addition, the third catheter 28 may have a soft tip having a flexible distal end part like a guiding catheter for a coronary artery.

The third catheter body 40 is inserted into the first lumen 241 of the first catheter 24. The third catheter body 40 is movable along the first lumen 241.

The third lumen 281 is disposed inside the third catheter body 40. The third lumen 281 extends along the third catheter body 40. The second catheter body 36 is movably provided in the third lumen 281. The proximal end of the third catheter body 40 includes a hub (not illustrated), and the proximal end of the hub is opened. The second catheter 26 can be inserted into the third lumen 281 through the hub.

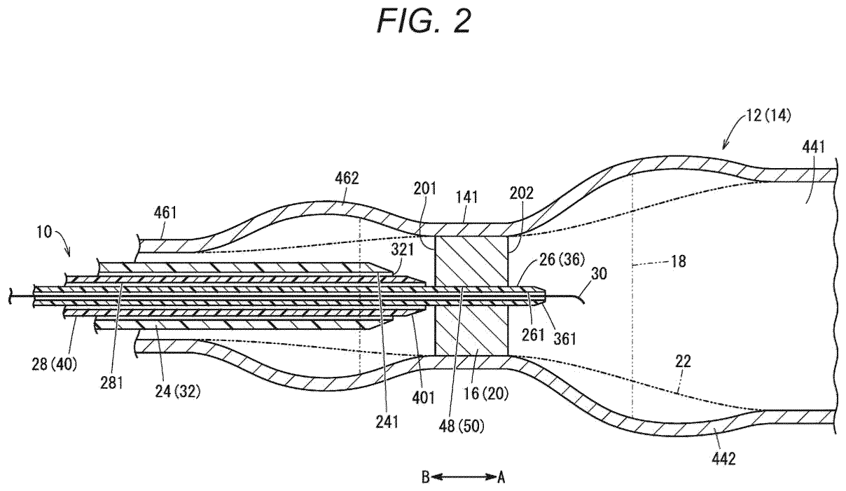

When the second catheter 26 moves along the third lumen 281 of the third catheter 28, the second catheter 26 is guided (supported) in the moving direction by the third catheter 28. The distal end part 401 of the third catheter 28 can protrude in the distal direction from the distal end part 321 of the first catheter 24 (see FIG. 2). That is, the radially outer side of the second catheter 26 is supported by the third catheter 28. In other words, the third catheter body 40 is disposed between the first lumen 241 of the first catheter body 32 and the outer peripheral surface of the second catheter body 36.

The venous thrombus treatment catheter 10 is not limited to the case of including three catheters (first to third catheters 24, 26, and 28). The third catheter 28 may not be provided, the first and second catheters 24 and 26 may be provided, and the second catheter body 36 may be directly inserted into the first lumen 241 of the first catheter body 32.

Next, a case where deep venous thrombosis (DVT) is treated using the venous thrombus treatment catheter 10 will be described. FIG. 1 is a schematic cross-sectional view illustrating a peripheral edge portion of the organized thrombus 20 (lesion 16) in a case where the organized thrombus 20 is generated in the blood vessel 141 of the vein 14 in a lower limb.

As illustrated in FIG. 1, in the vein 14 of the living body 12, a thrombus 18 is generated in the blood vessel 141, and a part of the thrombus 18 is organized to form the organized thrombus 20 (lesion 16). The right side of the organized thrombus 20 is the central side (heart side) and the downstream side of the blood flow. The left side of the organized thrombus 20 is the peripheral side (ankle side) and the upstream side of the blood flow. Hereinafter, the blood vessel 141 on the downstream side (right side) with respect to the organized thrombus 20 is referred to as a central blood vessel portion 441, and the blood vessel 141 on the upstream side (left side) with respect to the organized thrombus 20 is referred to as a peripheral blood vessel portion 461. In the central blood vessel portion 441, the diameter (both inner and outer diameter) of the blood vessel 141 is relatively larger than that of the peripheral blood vessel portion 461. The peripheral blood vessel portion 461 is a peripheral blood vessel having a relatively smaller diameter (both inner and outer diameter) of the blood vessel 141 than the central blood vessel portion 441.

In the peripheral blood vessel portion 461 on the peripheral side with respect to the organized thrombus 20 (lesion 16), blood flow is stagnated by the thrombus 18 (organized thrombus 20), and the blood vessel 141 is pressed radially outward by the blood to form a peripheral enlarged diameter portion 462 enlarged in diameter (both inner and outer diameter). The peripheral enlarged diameter portion 462 is enlarged in diameter (both inner and outer diameter) with respect to the diameter (both inner and outer diameter) of the blood vessel 141 (hereinafter, normal blood vessel 22) in the normal state in which the thrombus 18 is not formed or is not present (blood vessel devoid of the thrombus/lesion). The peripheral enlarged diameter portion 462 is formed on the upstream side of the organized thrombus 20. The peripheral enlarged diameter portion 462 is formed in a predetermined range in the extending direction of the blood vessel 141 (directions of arrows A and B).

In the central blood vessel portion 441 that is on the central side with respect to the organized thrombus 20 (lesion 16), a central enlarged diameter portion 442 is formed in which blood flow is stagnated by the thrombus 18 (organized thrombus 20) and the blood vessel 141 is pressed radially outward by the blood to be enlarged. The diameter of the central enlarged diameter portion 442 is enlarged with respect to the diameter of the normal blood vessel 22 in which the thrombus 18 is not formed. The central enlarged diameter portion 442 is formed on the downstream side of the organized thrombus 20. The central enlarged diameter portion 442 is formed in a predetermined range in the extending direction of the blood vessel 141 (directions of arrows A and B). That is, in the extending direction of the blood vessel 141 of the vein 14 in the living body 12, the organized thrombus 20 (lesion 16) is sandwiched between vein portions (peripheral enlarged diameter portion 462, central enlarged diameter portion 442) having diameters larger than the diameter of the normal blood vessel 22.

Hereinafter, as illustrated in FIG. 1, a case will be described in which the venous thrombus treatment catheter 10 is inserted from the peripheral blood vessel portion 461 on the peripheral side with respect to the organized thrombus 20 to treat the organized thrombus 20 (lesion 16).

First, a medical worker (not illustrated) identifies the position of the organized thrombus 20 in the vein 14 of the living body 12 by, for example, X-ray imaging, and then inserts the distal end part 321 of the first catheter 24 of the venous thrombus treatment catheter 10 percutaneously into the peripheral blood vessel portion 461 of the blood vessel 141. The method for identifying the position of the organized thrombus 20 is not limited to the X-ray imaging. Other methods may be used to identify the position of the organized thrombus 20 in the living body 12. Hereinafter, a case where the lesion 16 is treated under X-ray imaging will be described.

In the peripheral blood vessel portion 461, the distal end part 321 of the first catheter 24 is advanced toward the central side (direction of arrow A) toward the organized thrombus 20 along a guide wire (not illustrated), and the distal end part 321 of the first catheter 24 is placed on the upstream side of the organized thrombus 20. At this time, a medical worker (not illustrated) can perform a procedure while visually recognizing the position of the distal end part 321 of the first catheter 24 by visually recognizing a marker (not illustrated) arranged at the distal end part 321 of the first catheter body 32 through a display or the like under X-ray imaging.

Next, the distal end part 401 of the third catheter 28 is inserted into the first lumen 241 from the proximal end part (hub) of the first catheter 24, and the distal end part 401 is advanced toward the distal end part 321 of the first catheter 24 under X-ray imaging. At this time, the outer peripheral surface of the third catheter 28 is supported by the first catheter 24, and the third catheter 28 is guided in the distal direction. The distal end part 401 of the third catheter 28 is placed inside the distal end part 321 of the first catheter 24.

Next, the distal end part 361 of the second catheter 26 is inserted into the third lumen 281 from the proximal end part (hub) of the third catheter 28, and the distal end part 361 is advanced toward the distal end part 321 of the first catheter 24 and the distal end part 401 of the third catheter 28 under X-ray imaging. At this time, the outer peripheral surface of the second catheter 26 (second catheter body 36) is supported by the third catheter 28, and the second catheter 26 is guided in the distal direction. The distal end part 361 of the second catheter 26 is placed inside the distal end part 321 of the first catheter 24. The guide wire 30 is inserted through the second lumen 261 from the proximal end part (hub) of the second catheter 26. The distal end of the guide wire 30 is advanced to the distal end part 361 of the second catheter 26.

As illustrated in FIG. 2, the second and third catheters 26 and 28 are advanced in the distal direction (direction of arrow A) with respect to the first catheter 24, and the distal end part 361 of the second catheter 26 and the distal end part 401 of the third catheter 28 are inserted into the peripheral blood vessel portion 461 by protruding in the distal direction from the distal end part 321 of the first catheter 24.

By further advancing the second catheter 26 in the distal direction with respect to the third catheter 28, the distal end part 361 of the second catheter 26 together with the guide wire 30 protrudes in the distal direction (central side) from the distal end part 401 of the third catheter 28. At this time, the outer peripheral surface of the second catheter 26 is supported by the third catheter 28 in the traveling direction of the second catheter 26, and the second catheter 26 is guided in the distal direction.

In a state where the distal end of the guide wire 30 protrudes from the distal end part 361 of the second catheter 26 in the distal direction (direction of arrow A), the distal end part 361 of the second catheter 26 is caused to advance toward the organized thrombus 20 toward the central side (direction of arrow A) along the guide wire 30. By further moving the distal end part 361 of the second catheter 26 in the distal direction, the guide wire 30 comes into contact with and punctures an upstream end portion 201 of the organized thrombus 20, and then the distal end part 361 advances into the organized thrombus 20. As the distal end part 361 of the second catheter 26 advances to the central blood vessel portion 441, the organized thrombus 20 is dug by the distal end part 361 (the distal end part 361 digs into the organized thrombus 20), or a penetrating portion 48 is formed inside the organized thrombus 20 through the gap of the organized thrombus 20 (i.e., the distal end part 361 penetrates through the organized thrombus 20). The penetrating portion 48 is a perforation 50 penetrating or passing through the organized thrombus 20 along the extending direction (directions of arrows A and B) of the blood vessel 141. The penetrating portion 48 penetrates from the upstream end portion 201 to a downstream end portion 202 of the organized thrombus 20. At this time, the outer peripheral surface of the second catheter 26 is supported by the third catheter 28 in the traveling direction of the second catheter 26, and the second catheter 26 is guided in the distal direction.

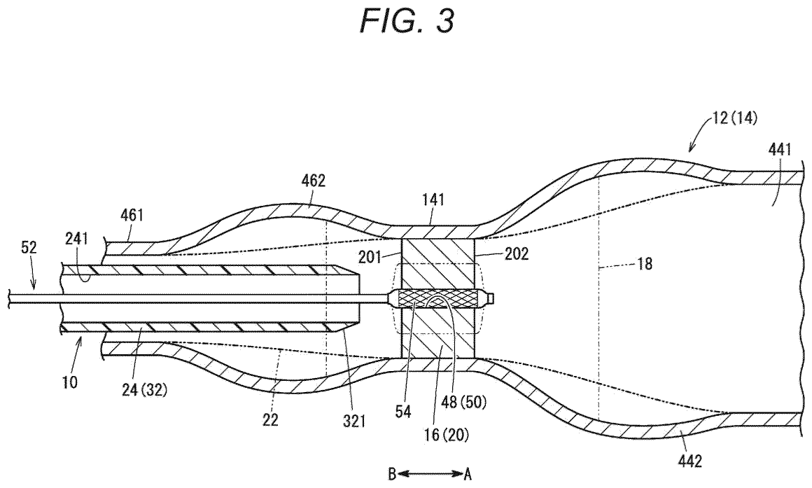

As illustrated in FIG. 3, after the penetrating portion 48 is formed in the organized thrombus 20, the second catheter 26 is removed from the penetrating portion 48. After the second and third catheters 26 and 28 are removed from the first catheter 24, the distal end part of the treatment catheter 52 is inserted into the first lumen 241 of the first catheter 24 from a hub (not illustrated). The treatment catheter 52 is, for example, a catheter having a treatment unit 54 expandable by operation of a medical worker at a distal end part. The treatment unit 54 is, for example, a balloon, a stent, or an atherectomy catheter. After the treatment unit 54 of the treatment catheter 52 moves along the first lumen 241 and protrudes in the distal direction from the distal end part 321 of the first catheter 24, the treatment unit 54 is inserted into the penetrating portion 48 (perforation 50) of the organized thrombus 20. When the treatment unit 54 is operated in the penetrating portion 48, the treatment unit 54 is expanded, and the organized thrombus 20 is pushed and spread to perform treatment (in FIG. 3, see two-dot chain line shape). After the penetrating portion 48 is formed in the organized thrombus 20, only the second catheter 26 may be removed from the first catheter 24, and the treatment catheter 52 may be inserted into the third lumen 281 of the third catheter 28 and advanced.

The method for treating the organized thrombus 20 is not limited to the case where only the second catheter 26 penetrates the organized thrombus 20. Since the outer diameter D1 of the first catheter 24 is smaller than the outer diameter D of the organized thrombus 20 (D1<D), the distal end part 321 of the first catheter 24 may advance into the organized thrombus 20 prior to the second catheter 26 to form the penetrating portion 48. Since the outer diameter D3 of the third catheter 28 is smaller than the outer diameter D1 of the first catheter 24, the distal end part 401 of the third catheter 28 may advance to the inside of the organized thrombus 20 prior to the second catheter 26 to form the penetrating portion 48.

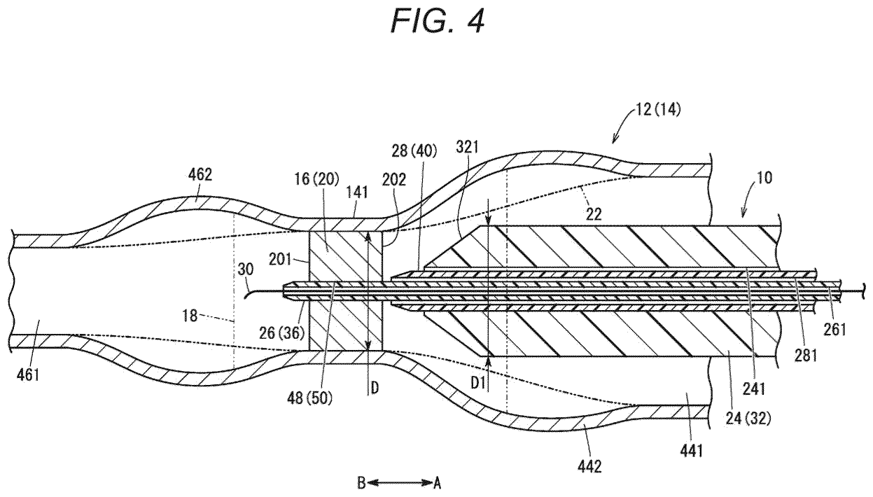

The treatment of the organized thrombus 20 using the venous thrombus treatment catheter 10 is not limited to the case where the venous thrombus treatment catheter 10 is inserted from the peripheral blood vessel portion 461. For example, as illustrated in FIG. 4, the treatment may be performed by inserting the venous thrombus treatment catheter 10 from the central blood vessel portion 441 which is the central side with respect to the organized thrombus 20. In this case, it is possible to use the venous thrombus treatment catheter 10 in which the outer diameter D1 of the first catheter 24 is larger than the outer diameter D of the organized thrombus 20 (D1>D). This is because the diameter of the blood vessel 141 in the central blood vessel portion 441 is larger than the diameter of the blood vessel 141 in the peripheral blood vessel portion 461.

The present embodiment has the following effects.

As illustrated in FIG. 1, the venous thrombus treatment catheter 10 is used to treat the lesion 16 having the organized thrombus 20 sandwiched between vein portions (central enlarged diameter portion 442, peripheral enlarged diameter portion 462) having diameters larger than the diameter of the blood vessel 141 in an extending direction (directions of arrows A and B) of the normal blood vessel 22 of the vein 14 in the living body 12. The venous thrombus treatment catheter 10 includes the first catheter 24 that has the first lumen 241 and punctures the skin of the living body 12, and the second catheter 26 that is movably inserted into the first lumen 241 of the first catheter 24, has the outer diameter D2 smaller than the outer diameter D of the organized thrombus 20, and is capable of protruding in the distal direction from the distal end part 321 of the first catheter 24.

According to this configuration, when the venous thrombus treatment catheter 10 is advanced into the vein 14 and the lesion 16 (organized thrombus 20) is punctured with the second catheter 26, the second catheter 26 is suitably backed up by the first catheter 24, and thus the second catheter 26 can be easily penetrated through the lesion 16 (organized thrombus 20). As a result, the treatment time of the lesion 16 by the venous thrombus treatment catheter 10 can be shortened. As compared with the case of a single catheter, by increasing the outer diameter D1 of the first catheter 24 to increase the rigidity and guiding the second catheter 26 by the first catheter 24, the second catheter 26 can be stably moved straight along the traveling direction.

By forming the outer diameter D1 of the first catheter 24 to be smaller than the outer diameter D of the organized thrombus 20, in a case where the venous thrombus treatment catheter 10 is inserted into the vein 14 from the peripheral side with respect to the lesion 16 of the living body 12, the first catheter 24 having a smaller diameter than the organized thrombus 20 can penetrate the lesion 16 (organized thrombus 20) together with the second catheter 26.

As illustrated in FIG. 4, by forming the outer diameter D1 of the first catheter 24 to be larger than the outer diameter D of the organized thrombus 20, the rigidity of the first catheter 24 is further enhanced, and the venous thrombus treatment catheter 10 can be inserted into the vein 14 from the central side where the diameter of the blood vessel 141 is larger than that of the terminal side, so that the support force of the second catheter 26 by the first catheter 24 can be further enhanced.

As illustrated in FIG. 1, the venous thrombus treatment catheter 10 includes the third catheter 28 that is movably inserted into the first lumen 241 of the first catheter 24, has the third lumen 281 into which the second catheter 26 is inserted, and is capable of protruding in a distal direction from the distal end part 321 of the first catheter 24. As a result, by guiding the outside of the second catheter 26 by the third catheter 28, the deflection of the second catheter 26 is suitably suppressed, and the support force of the second catheter 26 is improved, so that the penetration of the second catheter 26 with respect to the lesion 16 can be improved.

In the treatment method using the venous thrombus treatment catheter 10, the lesion 16 having the organized thrombus 20 in the vein 14 is identified, the venous thrombus treatment catheter 10 is advanced along the vein 14, and after the distal end of the venous thrombus treatment catheter 10 reaches the lesion 16, at least the second catheter 26 of the first and second catheters 24 and 26 is caused to penetrate the lesion 16.

According to this treatment method, when the venous thrombus treatment catheter 10 is advanced into the vein 14 and the lesion 16 is punctured with the second catheter 26, the second catheter 26 is suitably backed up by the first catheter 24, so that the second catheter 26 can easily penetrate the lesion 16 (organized thrombus 20). As a result, the treatment time of the lesion 16 by the venous thrombus treatment catheter 10 can be shortened. As compared with the case of a single catheter, by increasing the outer diameter D1 of the first catheter 24 to further increase the rigidity and guiding the second catheter 26 by the first catheter 24, the second catheter 26 can be stably moved straight along the traveling direction.

After the penetrating portion 48 is formed in the lesion 16 by penetrating at least the second catheter 26 through the lesion 16, the treatment catheter 52 is inserted into the penetrating portion 48 of the lesion 16 to treat the lesion 16, whereby the penetrating portion 48 is formed with respect to the lesion 16 by the second catheter 26, whereby the treatment catheter 52 is inserted into the penetrating portion 48 and the treatment of the lesion 16 can be reliably performed by the treatment catheter 52.

The detailed description above describes embodiments of a venous thrombus treatment catheter and operational method representing examples of the new venous thrombus treatment catheter and operation disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A venous thrombus treatment catheter for treating, in a blood vessel of a vein in a living body, a lesion having an organized thrombus sandwiched between vein portions in an extending direction of the blood vessel, with the vein portions having diameters larger than a diameter of the blood vessel devoid of the thrombus, the venous thrombus treatment catheter comprising:
a first catheter having a first lumen and a distal end configured to puncture skin of the living body; and
a second catheter that is movably insertable into the first lumen of the first catheter, has an outer diameter smaller than an outer diameter of the organized thrombus, and is configured to protrude in a distal direction from a distal end part of the first catheter.

2. The venous thrombus treatment catheter according to claim 1, wherein an outer diameter of the first catheter is smaller than an outer diameter of the organized thrombus.

3. The venous thrombus treatment catheter according to claim 1, wherein an outer diameter of the first catheter is larger than an outer diameter of the organized thrombus.

4. The venous thrombus treatment catheter according to claim 1, comprising:
a third catheter that is movably insertable into the first lumen of the first catheter, has a third lumen into which the second catheter is insertable, and is configured to protrude in a distal direction from a distal end part of the first catheter.

5. The venous thrombus treatment catheter according to claim 1, wherein the second catheter has a second lumen, and a guide wire is insertable through the second lumen.

6. The venous thrombus treatment catheter according to claim 1, wherein the distal end part of the first catheter has a tapered outer peripheral surface in which an outer diameter of the first catheter decreases in a distal direction, and wherein a distal end part of the second catheter has a tapered outer peripheral surface in which the outer diameter of the second catheter decreases in the distal direction.

7. The venous thrombus treatment catheter according to claim 6, comprising a third catheter that is movably insertable into the first lumen of the first catheter and that is configured to protrude in the distal direction from the distal end part of the first catheter.

8. The venous thrombus treatment catheter according to claim 7, wherein a distal end part of the third catheter has a tapered outer peripheral surface in which an outer diameter of the third catheter decreases in the distal direction.

9. The venous thrombus treatment catheter according to claim 6, comprising a third catheter that is movably insertable into the first lumen of the first catheter and that has a third lumen in which the second catheter is positionable such that the distal end part of the second catheter protrudes in the distal direction beyond a distal end part of the third catheter while the distal end part of the third catheter protrudes in the distal direction beyond the distal end part of the first catheter.

10. A treatment method for treating, by a venous thrombus treatment catheter, a lesion in a blood vessel of a vein in a living body, the lesion having an organized thrombus sandwiched between vein portions in an extending direction of the blood vessel of the vein in the living body, the vein portions between which is sandwiched the organized thrombus having diameters larger than a diameter of the blood vessel devoid of the thrombus,
the venous thrombus treatment catheter including:
a first catheter having a first lumen and configured to puncture skin of the living body; and
a second catheter that is movably positioned in the first lumen of the first catheter, has an outer diameter smaller than an outer diameter of the organized thrombus, and is configured to protrude in a distal direction from a distal end part of the first catheter, and
the treatment method comprising:
identifying the lesion with the organized thrombus; and
causing the venous thrombus treatment catheter to advance along the vein, and causing at least the second catheter that is positioned in the first lumen of the first catheter to penetrate the lesion after a distal end of the venous thrombus treatment catheter reaches the lesion.

11. The treatment method according to claim 10, wherein the causing of the second catheter to penetrate the lesion produces a penetrating portion in the lesion that penetrates through the lesion, the method further comprising:
after forming the penetrating portion in the lesion by the penetrating of the at least the second catheter into the lesion, inserting a treatment catheter into the penetrating portion of the lesion to treat the lesion.

12. The treatment method according to claim 10, wherein the venous thrombus treatment catheter includes a third catheter positioned inside the first catheter, the third catheter including a third lumen extending through the third catheter, the second catheter being positioned inside the third lumen of the third catheter,
the causing of the venous thrombus treatment catheter to advance along the vein including causing the venous thrombus treatment catheter to advance along the vein while the third catheter is positioned inside the first lumen of the first catheter and while the second catheter is positioned inside the third lumen of the third catheter.

13. The treatment method according to claim 10, wherein the causing of the second catheter to penetrate the lesion produces a penetrating portion passing through the lesion, the method further comprising:
after forming the penetrating portion in the lesion by the penetrating of the at least the second catheter into the lesion, removing the second catheter from the first catheter;
inserting a treatment catheter into the penetrating portion of the lesion; and
treating the lesion through operation of the treatment catheter.

14. The treatment method according to claim 13, wherein the inserting of the treatment catheter into the penetrating portion of the lesion occurs after removing the second catheter from the first catheter.

15. The treatment method according to claim 13, wherein the inserting of the treatment catheter into the penetrating portion of the lesion includes conveying the treatment catheter through the first lumen of the first catheter before inserting the treatment catheter into the penetrating portion of the lesion.

16. The treatment method according to claim 13, wherein the causing of the venous thrombus treatment catheter to advance along the vein includes causing the venous thrombus treatment catheter to advance toward the lesion from an upstream side of blood flow in the vein toward a downstream side of blood flow in the vein.

17. A venous thrombus treatment catheter for treating, in a blood vessel of a vein in a living body, a lesion having an organized thrombus sandwiched between vein portions in an extending direction of the blood vessel, with the vein portions having diameters larger than a diameter of the blood vessel devoid of the thrombus, the venous thrombus treatment catheter comprising:
a combination of a first catheter having a first lumen extending throughout the first catheter and a second catheter having a second lumen extending throughout the second catheter;
the second catheter being positioned in the first lumen of the first catheter and being movable relative to the first catheter from a position in which a most distal end of the first catheter is on a distal side of a most distal end of the second catheter to a position in which the most distal end of the second catheter is on the distal side of the most distal end of the first catheter so that the second catheter is able to penetrate into the lesion;
the first catheter having a distal end part that is tapered in a narrowing manner toward the most distal end of the first catheter so that the distal end part is configured to puncture skin of the living body; and
the second catheter having a distal end part that is tapered in a narrowing manner toward the most distal end of the second catheter.

18. The venous thrombus treatment catheter according to claim 17, comprising a third catheter having a third lumen and being movably positioned in the first lumen while the second catheter is movably positioned in the third lumen, the second catheter being configured to protrude in a distal direction beyond the most distal end of the first catheter.

19. The venous thrombus treatment catheter according to claim 18, wherein a distal end part of the third catheter has a tapered outer peripheral surface in which the outer diameter of the third catheter decreases in the distal direction.
